(19) United States
(12) Patent Application Publication
(10) Pub. No.: US 2020/0390610 A1
(43) Pub. Date: Dec. 17, 2020

(54) Wound Covering Device

(71) Applicant: Sidney Bowman, Sharpsville, IN (US)

(72) Inventor: Sidney Bowman, Sharpsville, IN (US)

(21) Appl. No.: 16/437,779

(22) Filed: Jun. 11, 2019

(51) Int. Cl. A61F 13/02; A61F 13/00

(52) U.S. Cl. CPC A61F 13/025; A61F 13/0233; A61F 2013/00165; A61F 2013/00217; A61F 2013/00182; A61F 13/00085

(57) ABSTRACT

A wound covering device for noncontact wound protection includes a shell and a coupler. The shell, which defines an interior space, has a bottom that is open so that the shell is configured to position over a wound in skin of a user. The coupler is coupled to the shell and is configured to removably couple to the skin of the user. The coupler is configured to retain the shell over the wound to protect the wound.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR Not Applicable

### BACKGROUND OF THE INVENTION

### (1) Field of the Invention

### (2) Description of Related Art Including Information Disclosed under 37 CFR 1.97 and 1.98

The disclosure and prior art relate to wound dressings and more particularly pertain to a new wound dressings for noncontact wound protection.

### BRIEF SUMMARY OF THE INVENTION

An embodiment of the disclosure meets the needs presented above by generally comprising a shell and a coupler. The shell, which defines an interior space, has a bottom that is open so that the shell is configured to position over a wound in skin of a user. The coupler is coupled to the shell and is configured to removably couple to the skin of the user. The coupler is configured to retain the shell over the wound to protect the wound.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a wound covering device according to an embodiment of the disclosure.

FIG. 2 is a bottom view of an embodiment of the disclosure.

FIG. 3 is a top view of an embodiment of the disclosure.

FIG. 4 is an in-use view of an embodiment of the disclosure.

FIG. 5 is a bottom view of an embodiment of the disclosure.

FIG. 6 is an isometric perspective view of an embodiment of the disclosure.

### DETAILED DESCRIPTION OF THE INVENTION

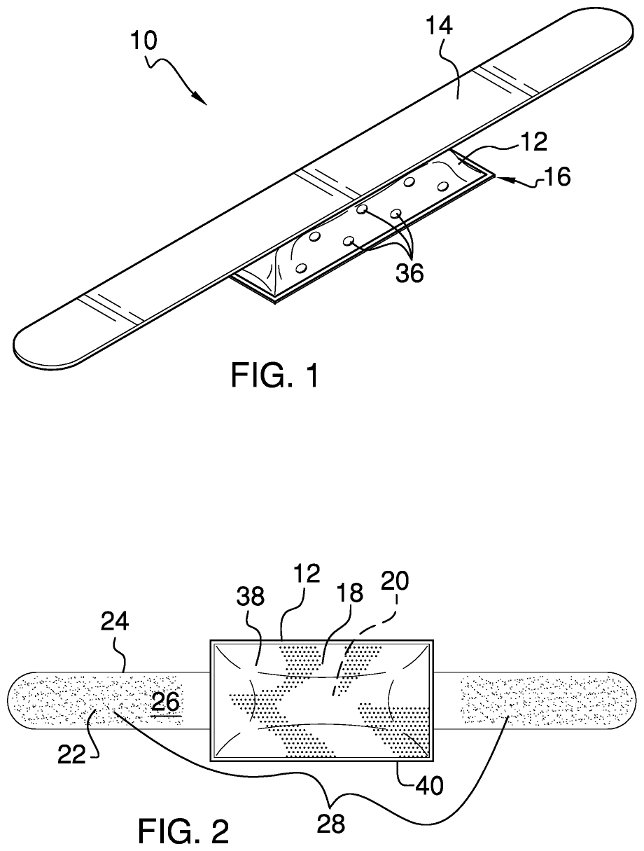

With reference now to the drawings, and in particular to FIGS. 1 through 6 thereof, a new wound dressings embodying the principles and concepts of an embodiment of the disclosure and generally designated by the reference numeral 10 will be described.

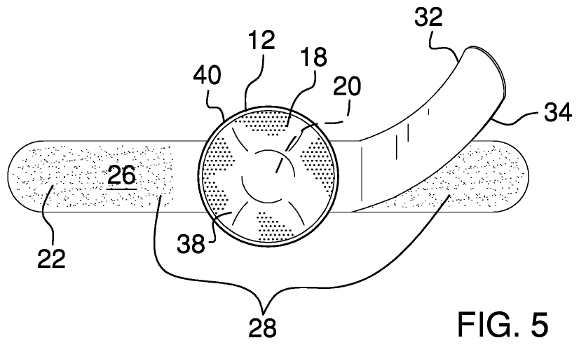

As best illustrated in FIGS. 1 through 6, the wound covering device 10 generally comprises a shell 12 and a coupler 14. The shell 12, which defines an interior space 16, has a bottom 18 that is open so that the shell 12 is configured to position over a wound in skin of a user. The shell 12 comprises at least one of rubber, silicone, and plastic. The bottom 18 is at least one of rectangularly shaped (FIG. 2), squarely shaped (not shown), ovally shaped (not shown), and circularly shaped (FIG. 5). A top 20 of the shell 12 may be circumferentially smaller than the bottom 18 of the shell 12, as shown in FIG. 5.

The coupler 14 is coupled to the shell 12 and is configured to removably couple to the skin of the user so that the coupler 14 is configured to retain the shell 12 over the wound to protect the wound.

The coupler 14 comprises an adhesive 22 that is configured to removably couple to the skin of the user as the shell 12 is positioned over the wound. The adhesive 22 is pressure sensitive type so that the adhesive 22 is configured to bond under application of light pressure.

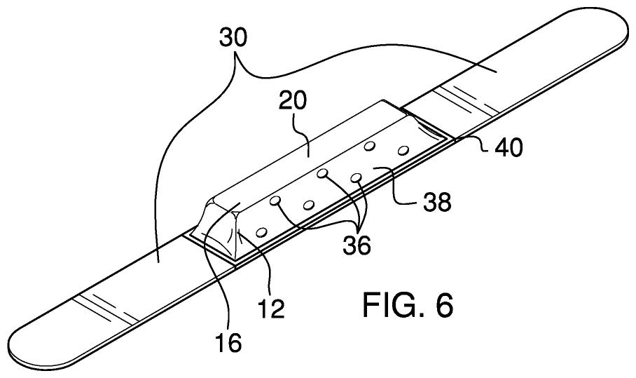

In one embodiment, as shown in FIG. 1, the coupler 14 comprises a strap 24 that is coupled to the top 20 of the shell 12 and which extends bidirectionally from the shell 12. The strap 24 is flexible. The adhesive 22 is positioned on a lower face 26 of the strap 24 on opposing end sections 28 of the strap 24. The adhesive 22 is configured to removably couple to the skin of the user so that the strap 24 exerts a downward force on the shell 12 to sealably couple the bottom 18 of the shell 12 to the skin. The strap 24 comprises at least one of fabric, polyvinylchloride, polyethylene, and polyurethane so that the strap 24 is flexible. The strap 24 may be narrower than the top 20 of the shell 12, as shown in FIG. 2, substantially as wide as the bottom 18 of the shell 12, as shown in FIG. 6, or wider than the top 20 of the shell 12 (not shown).

In another embodiment, as shown in FIG. 6, the strap 24 comprises a pair of strips 30. Each strip 30 is coupled to and extends from the shell 12 proximate to the bottom 18 so that the strips 30 are substantially opposingly positioned on the shell 12.

A panel 32 is removably coupled to the adhesive 22 so that the panel 32 is selectively detachable from the adhesive 22. The panel 32 is configured to prevent unintended adherence of the adhesive 22. The panel 32 comprises at least one of paper and plastic. A release agent 34 is positioned on the panel 32 so that the panel 32 is configured to removably coupled to the adhesive 22.

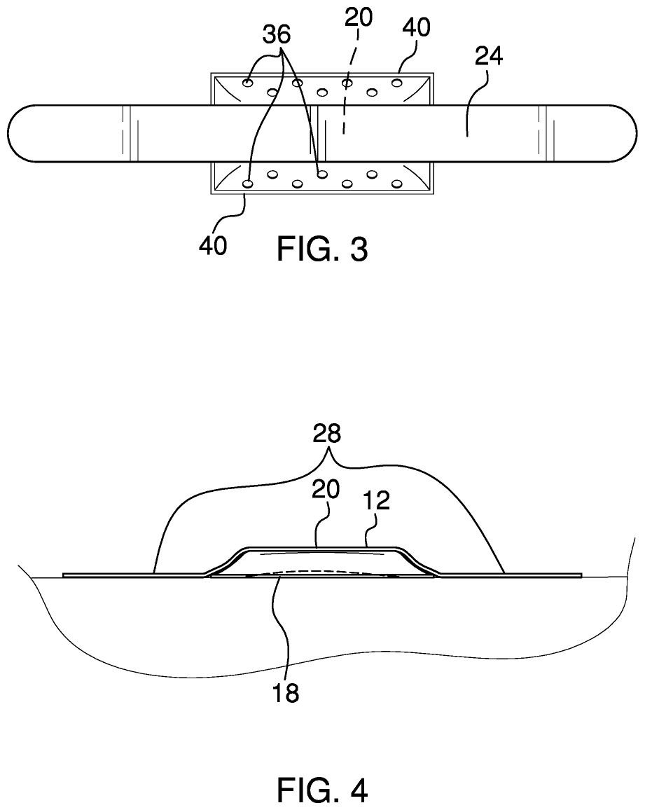

As shown in FIG. 3, a plurality of holes 36 may be positioned in a sidewall 38 of the shell 12 to vent the interior space 16. Venting the interior space 16 serves to dry out the wound. When the shell 12 lack the plurality of holes 36, as shown in FIG. 4, the shell 12 is occlusive and may be used to retain a medicant between the shell 12 and the wound.

A lip 40 is coupled to and extends from the sidewall 38 adjacent to the bottom 18. The lip 40 is configured to distribute a force applied by the shell 12 to the skin to increase comfort of the user. The lip 40 extends outwardly from the shell 12.

In use, the panel 32 is decoupled from the adhesive 22 so that the user is positioned to place the shell 12 over the wound and to press down on the opposing end sections 28 of the strap 24 to couple the shell 12 to the skin. The shell 12 thus is configured to protect the wound.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be only one of the elements.

I claim:

1. A wound covering device comprising:
a shell defining an interior space, the shell having a bottom, the bottom being open wherein the shell is configured for positioning over a wound in skin of a user; and
a coupler coupled to the shell, the coupler being configured for removably coupling to the skin of the user wherein the coupler is configured for retaining the shell over the wound for protecting the wound.

2. The device of claim 1, further including the shell comprising at least one of rubber, silicone, and plastic.

3. The device of claim 1, further including the bottom being at least one of rectangularly shaped, squarely shaped, ovally shaped, and circularly shaped.

4. The device of claim 1, further including the coupler comprising an adhesive wherein the adhesive is configured for removably coupling to the skin of the user as the shell is positioned over the wound.

5. The device of claim 4, further including the adhesive being pressure sensitive type such that the adhesive is configured for bonding under application of light pressure.

6. The device of claim 4, further including a strap coupled to a top of the shell and extending bidirectionally from the shell, the strap being flexible, the adhesive being positioned on a lower face of the strap on opposing end sections of the strap wherein the adhesive is configured for removably coupling to the skin of the user such that the strap exerts a downward force on the shell for sealably coupling the bottom of the shell to the skin.

7. The device of claim 6, further including the strap comprising at least one of fabric, polyvinylchloride, polyethylene, and polyurethane such that the strap is flexible.

8. The device of claim 6, further including the strap comprising a pair of strips, each strip being coupled to and extending from the shell proximate to the bottom such that the strips are substantially opposingly positioned on the shell.

9. The device of claim 6, further including a panel removably coupled to the adhesive such that the panel is selectively detachable from the adhesive wherein the panel is configured for preventing unintended adherence of the adhesive.

10. The device of claim 9, further including the panel comprising at least one of paper and plastic.

11. The device of claim 9, further including a release agent positioned on the panel such that the panel is configured for removably coupling to the adhesive.

12. The device of claim 1, further including a plurality of holes positioned in a sidewall of the shell wherein the holes are configured for venting the interior space.

13. The device of claim 1, further including a lip coupled to and extending from a sidewall adjacent to the bottom wherein the lip is configured for distributing a force applied by the shell to the skin for increasing comfort of the user.

14. The device of claim 13, further including the lip extending outwardly from the shell.

15. A wound covering device comprising:
a shell defining an interior space, the shell having a bottom, the bottom being open wherein the shell is configured for positioning over a wound in skin of a user, the shell comprising at least one of rubber, silicone, and plastic, the bottom being at least one of rectangularly shaped, squarely shaped, ovally shaped, and circularly shaped;
a coupler coupled to the shell, the coupler being configured for removably coupling to the skin of the user wherein the coupler is configured for retaining the shell over the wound for protecting the wound, the coupler comprising an adhesive wherein the adhesive is configured for removably coupling to the skin of the user as the shell is positioned over the wound, the adhesive being pressure sensitive type such that the adhesive is configured for bonding under application of light pressure;
a strap coupled to a top of the shell and extending bidirectionally from the shell, the strap being flexible, the adhesive being positioned on a lower face of the strap on opposing end sections of the strap wherein the adhesive is configured for removably coupling to the skin of the user such that the strap exerts a downward force on the shell for sealably coupling the bottom of the shell to the skin, the strap comprising at least one of fabric, polyvinylchloride, polyethylene, and polyurethane such that the strap is flexible; the strap comprising a pair of strips, each strip being coupled to and extending from the shell proximate to the bottom such that the strips are substantially opposingly positioned on the shell;
a panel removably coupled to the adhesive such that the panel is selectively detachable from the adhesive wherein the panel is configured for preventing unintended adherence of the adhesive, the panel comprising at least one of paper and plastic;
a release agent positioned on the panel such that the panel is configured for removably coupling to the adhesive;
a plurality of holes positioned in a sidewall of the shell wherein the holes are configured for venting the interior space; and
a lip coupled to and extending from the sidewall adjacent to the bottom wherein the lip is configured for distributing a force applied by the shell to the skin for increasing comfort of the user, the lip extending outwardly from the shell.
